(19) United States
(12) Patent Application Publication
(10) Pub. No.: US 2019/0240374 A1
(43) Pub. Date: Aug. 8, 2019

(54) DEGRADABLE BULK METALLIC MAGNESIUM/POLYMER COMPOSITE BARRIER MEMBRANES FOR DENTAL, CRANIOMAXILLOFACIAL AND ORTHOPEDIC APPLICATIONS AND MANUFACTURING METHODS

(71) Applicant: UNIVERSITY OF PITTSBURGH-OF THE COMMONWEALTH SYSTEM OF HIGHER EDUCATION, PITTSBURGH, PA (US)

(72) Inventors: ANDREW BROWN, PITTSBURGH, PA (US); CHARLES SFEIR, PITTSBURGH, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 16/341,977

(22) PCT Filed: Oct. 23, 2017
(86) PCT No.: PCT/US17/57815

§ 371 Date: Apr. 15, 2019

Related U.S. Application Data
(60) Provisional application No. 62/411,288, filed on Oct. 21, 2016.

(51) Int. Cl. A61L 27/04; A61F 2/28; A61B 17/80; A61L 27/34; A61L 27/58; A61K 6/087; A61K 6/04

(52) U.S. Cl. CPC A61L 27/047; A61F 2/2846; A61B 17/8085; A61L 27/34; A61F 2310/00041; A61K 6/087; A61K 6/04; A61L 2420/02; A61L 2420/08; A61L 27/58

(57) ABSTRACT

The invention relates to magnesium reinforcements and magnesium-reinforced barrier membranes for use in biomedical applications, such as dental, craniofacial and orthopedic applications. The magnesium reinforcements and barrier membranes are composed of a biodegradable, magnesium/polymer composite. They can be used in a wide variety of applications, such as, but not limited to, vertical and horizontal ridge augmentation, guided bone/tissue regeneration, periodontal bone regeneration, fracture fixation and orthopedic and spinal bone grafting applications; as well as in general surgery (hernia repair) and urogynecological surgery.

### CROSS-REFERENCE TO RELATED APPLICATIONS

This application claims priority under 35 U.S.C. § 119(e) to U.S. Provisional Patent Application Ser. No. 62/411,288, filed Oct. 21, 2016, entitled “Degradable Bulk Metallic Magnesium/Polymer Composite Barrier Membranes for Dental, Craniomaxillofacial and Orthopedic Applications and Manufacturing Methods”, which is herein incorporated by reference.

### GOVERNMENT SUPPORT

This invention was made with government support under EEC0812348 and IIP1449702 awarded by the National Science Foundation (NSF). The government has certain rights in the invention.

### FIELD OF THE INVENTION

The invention relates to magnesium reinforcements and magnesium-reinforced barrier membranes for use in biomedical applications, such as dental, craniofacial and orthopedic applications. The magnesium reinforcements and magnesium-reinforced barrier membranes of the invention can be used in a wide variety of applications, such as, but not limited to, vertical and horizontal ridge augmentation, guided bone/tissue regeneration, periodontal bone regeneration, fracture fixation and orthopedic and spinal bone grafting applications; as well as in general surgery (hernia repair) and urogynecological surgery.

### BACKGROUND

Biomedical implant devices are known in the art and are commonly used in the practice of various surgeries, such as, orthopedic, dental, craniofacial and cardiovascular implant surgeries. These devices may be used for various purposes, including tissue and bone regeneration, and drug or biomolecule delivery. There are a wide variety of implant devices that include, but are not limited to, scaffolds, such as plates and screws, membranes, meshes, and the like.

Non-bioresorbable barrier membranes (e.g., titanium-reinforced, poly(tetrafluoroethylene), titanium micromesh, and the like) are commonly used to a) contain bone grafting material within a bone defect that is being regenerated, and b) protect a healing bone defect site from mechanical insults prior to dental implant placement. Unfortunately, since these membranes are non-degradable and reside under the oral mucosa, they must be removed following bone healing. Their removal requires a separate surgery with unnecessary anesthesia, potential risks of related infection, increased treatment costs, unnecessary pain experienced by the patient, as well as inconvenience to the patient. Additionally, the titanium-reinforced barrier membranes suffer from a high exposure and dehiscence rate that requires unpredictable clinical intervention.

Bioresorbable barrier membranes (e.g., collagen, poly(lactic-co-glycolic acid), and the like) are clinically used and are known to contain bone grafting material within a bone defect that is being regenerated. However, due to their low stiffness, they do not protect healing bone defect sites from mechanical insult. While the bioresorbable barrier membranes do not provide mechanical protection, they do eliminate the device removal surgery required of existing non-bioresorbable barrier membranes.

Titanium-reinforced membranes are also frequently used in the place of fixation plates to stabilize fractures occurring in bones with complex geometry, such as, the skull, orbital, mandible, and the like. The geometry of the membranes, e.g., meshes, can be designed such that they provide a snug fit over concave shapes that are typically difficult to fixate. Unfortunately, the titanium meshes are frequently removed following fracture healing, particularly in pediatric cases, because of their potential to interfere with normal bone growth.

Biomaterials for the construction of implant devices are typically chosen based on their ability to withstand cyclic load-bearing and compatibility with the physiological environment of a human body. Many of these implant devices are traditionally constructed of polymer or non-bioresorbable metal, e.g., titanium. These materials of construction exhibit good biomechanical properties. Non-bioresorbable metallic biomaterials, in particular, have appropriate properties such as high strength, ductility, fracture toughness, hardness, corrosion resistance, formability, and biocompatibility to make them attractive for most load bearing applications. Polymers, such as polyhydroxy acids, polylactic acid (PLA), polyglycolic acid (PGA), and the like, are known as conventional biomaterials, however, in some instances the strength and ductility exhibited by polymers is not as attractive as that demonstrated by metallic biomaterials. For example, it is known to use stainless steel or titanium biomedical implants for clinical applications which require load-bearing capacities.

Magnesium is potentially attractive as a biomaterial because it is very lightweight, has a density similar to cortical bone, has an elastic modulus close to natural bone, is essential to human metabolism, is a cofactor for many enzymes, and stabilizes the structures of DNA and RNA. Magnesium-based implants may be degradable in-vivo through simple corrosion and exhibit mechanical properties similar to native bone. Thus, magnesium can be used to provide mechanical properties approaching those of traditional non-bioresorbable metallic biomaterials (e.g., titanium, stainless steel, and the like) while providing the advantage of being bioresorbable.

There is a desire to design and develop bulk metallic magnesium/polymer barrier membranes that provide the same clinical utility, e.g., containment of grafting material and protection of a healing defect site from mechanical insults, while being implanted using the same tools and procedures that are employed for the traditional membranes, e.g., meshes, as well as being fully and safely degradable once implanted. Thus, eliminating the need for a second device removal surgery, while decreasing the rate of exposure and dehiscence.

Furthermore, it is an object of the invention to design and develop magnesium devices that can be designed in a pattern that optimizes geometric fit and degradation, while being degradable following bone healing which also eliminates the need for a second device removal surgery.

### SUMMARY OF THE INVENTION

An aspect of the invention provides a method of preparing a biodegradable, magnesium-reinforced polymer composite implant device. The method includes selecting bulk magnesium; selecting a polymer sheet; processing the bulk magnesium to form a pre-determined geometry having a surface, employing a process selected from the group consisting of expanded metal processing and laser cutting; and applying the polymer sheet onto the surface of the pre-determined geometry.

The predetermined geometry may be selected from a magnesium framework that allows adaptability of the membrane to a bone defect site and subsequent fixation. In certain embodiments, the predetermined geometry is selected from a mesh, strut and strut-style support. The bulk magnesium may be in a form selected from a foil and sheet.

The surface can include an upper surface and a lower surface, and the polymer sheet can be applied to one or more of the upper and lower surfaces. In certain embodiments, applying the polymer sheet includes melting a first polymer sheet onto the upper surface and a second polymer sheet onto the lower surface, wherein the first polymer sheet is the same as, or different from, the second polymer sheet.

The method can include obtaining a polymer in dry form and integrating the polymer in dry form into the pre-determined geometry, wherein the integrating is conducted prior to the melting of the first and second polymer sheets. In certain embodiments, the integrating includes embedding the polymer in dry form within a plane of a magnesium mesh having pores or open spaces formed therein. In other embodiments, the integrating includes depositing the polymer in dry form along a perimeter of a magnesium strut. The applying of the polymer sheet can employ a process selected from the group consisting of compression molding and laminating.

In another aspect, the invention provides a biodegradable, magnesium-reinforced polymer composite that includes a magnesium framework having a surface, in a form selected from the group consisting of mesh, strut and strut-style support, and a polymer sheet applied to the surface of the magnesium framework.

The mesh can include polymer powder embedded in pores and openings formed in the mesh. The strut can include polymer powder formed along a perimeter of the strut.

The polymer sheet may be selected from low molecular weight poly(lactic-co-glycolic acid), high molecular weight poly(lactic-co-glycolic acid), poly-L-lactic acid, poly-D-lactic acid, polyethylene glycol, and blends and mixtures thereof.

The composite may be a biomedical implant device for applications selected from the group consisting of dental, craniofacial and orthopedic applications. The applications may include containing bone graft material and fixating complex craniofacial bone fractures.

### DETAILED DESCRIPTION OF THE INVENTION

The invention relates generally to biodegradable, magnesium-reinforced polymer composites and, more particularly, to implant devices including magnesium reinforcements and magnesium-reinforced barrier membranes for use in biomedical applications, such as dental, craniofacial and orthopedic applications. The magnesium reinforcements and barrier membranes provide improvements over traditional titanium meshes and titanium-reinforced barrier membranes, which are employed for containing bone graft material or for fixating complex craniofacial bone fractures. The magnesium reinforcements and barrier membranes of the invention can be used in a wide variety of applications, such as, but not limited to, vertical and horizontal ridge augmentation, guided bone/tissue regeneration, periodontal bone regeneration, fracture fixation and orthopedic and spinal bone grafting applications; as well as in general surgery (hernia repair) and urogynecological surgery.

There is an interest in designing and developing biodegradable materials for reinforcements and barrier membranes because after a period of time, the implant device is no longer needed, e.g., after bone or tissue healing is complete. The device can be left in situ or, alternatively, can be removed. Each of these alternatives has disadvantages associated therewith. For example, leaving the device in situ increases the chances of infection and rejection, and removal of the device requires a second surgery and causes a risk of infection, pain and discomfort to the patient, as well as it being an additional expense. A resorbable implant device that is effective to degrade over a period of time, e.g., by dissolving in the physiological environment, can overcome the aforementioned disadvantages. Thus, the device does not remain in-situ and there is no need to surgically remove the device when the device is no longer needed. However, resorbable materials, such as, polymers, can lack mechanical strength as compared to that exhibited by metal implants. As a result, the combination of polymer with bulk metallic magnesium is advantageous.

Magnesium and its alloys have mechanical properties compatible to bone and tissue, and can be resorbed over a period of time. For example, magnesium is very lightweight, has a density similar to cortical bone, has an elastic modulus also close to natural bone, is essential to human metabolism, is a cofactor for many enzymes, and stabilizes the structures of DNA and RNA. As for the magnesium, it has been demonstrated in the art that this elemental metal and its alloys exhibit both biocompatibility and biodegradability. For example, magnesium and its alloys have been shown to promote both bone and cartilage. Further, it has been demonstrated that degrading magnesium scaffolds promote both bone formation and resorption.

In accordance with the invention, a magnesium reinforcement includes a magnesium framework that allows adaptability of the barrier membrane to a bone defect site and subsequent fixation. Non-limiting examples of magnesium reinforcements include, but are not limited to, meshes, struts and strut-style supports. The magnesium reinforcements are composed of, and prepared from, bulk magnesium or bulk magnesium alloy. The term “bulk” is used herein to indicate that the magnesium or magnesium alloy is in the form of a single mass, as compared to a plurality of particles or granules. For example, the magnesium or magnesium alloy for use in the invention can be in the form of a foil, sheet, membrane or the like, as compared to a powder form.

The magnesium-reinforced polymer barrier membranes can include polymer selected from the wide variety of polymers that are known in the art. The barrier membranes are inclusive of meshes. Non-limiting examples of suitable polymer for use in the invention include, but are not limited to, poly(lactic-co-glycolic acid), e.g., low molecular weight poly(lactic-co-glycolic acid) and/or high molecular weight poly(lactic-co-glycolic acid), poly(lactic-co-glycolic acid), poly-L-lactic acid, poly-D-lactic acid, polyethylene glycol, and blends and mixtures thereof. The polymer is selected to optimize handling and provide sufficient mechanical strength when the device is implanted in vivo.

In addition to selecting the magnesium and polymer components, a predetermined geometry may be selected for the magnesium framework that allows adaptability of the membranes to a bone defect site and subsequent fixation.

The magnesium reinforcements and magnesium-reinforced barrier membranes can be employed as substitutes for the traditional titanium reinforcements and titanium-reinforced membranes. Thus, the magnesium reinforcements and magnesium-reinforced barrier membranes are used in place of fixation plates to stabilize fractures occurring in bones with complex geometry. Further, the geometry of the membranes and meshes can be designed such that they provide a snug fit over concave shapes that are typically difficult to fixate. A difference, i.e., an advantage, between magnesium and titanium is that use of magnesium provides a degradable, implant device that does not require surgical removal as does the titanium-reinforced device.

Titanium barrier membranes are manufactured using a variety of known methods based on the particular properties of titanium. Magnesium barrier membranes and meshes can also be manufactured using known techniques. However, various machining and processing techniques used for titanium are not directly applicable to the use of magnesium due to the difference in properties between the materials. In certain embodiments, known laser cutting technology is used to form the magnesium reinforcements and magnesium-reinforced polymer barrier membranes from the bulk magnesium, e.g., a magnesium foil or magnesium sheet. Laser cutting technology includes a variety of cutting methods that use lasers. In general, the beam of a high-powered laser is directed, e.g., commonly through optics, at the material. As a result of the focused laser beam, the material then either melts, burns, vaporizes away, or is blow away by a jet of gas, leaving an edge with a high-quality surface finish. Accordingly, the magnesium foil or sheet is subjected to a focused laser beam for laser cutting a pre-determined geometry to produce meshes and strut-style supports, for example.

Following laser cutting to produce a mesh or strut-style support according to the invention, the reinforcement or barrier membrane undergoes a post-processing heat treatment.

Scaling-up manufacturing of the magnesium meshes or strut-style support can include production through an expanded metal process followed by heat treatment post-processing. Expanded metal technology is known in the art. In general, expanded metal is a type of sheet metal which can be cut and stretched to form a pattern of metal, such as, a mesh-like material. Expanded metal may be stronger than an equivalent weight of wire mesh because the material is flattened, allowing the metal to remain in a single piece.

In certain embodiments, magnesium-reinforced polymer barrier membranes are formed by embodying the bulk magnesium meshes within a degradable polymer matrix, e.g., PLGA matrix. This composite embodiment can be used in a variety of applications, e.g., surgeries, including periodontal, oral maxillofacial bone grafting, orthopedic and urogynecological.

The reinforcements and barrier membranes can be produced using a fabrication or manufacture procedure that includes a compression molding process or a lamination process. The steps in the procedure are described below.

Step 1. A magnesium foil/sheet is subjected to expanded metal processing or laser cutting to produce a magnesium mesh or magnesium strut of varying geometries. A particular, e.g., pre-determined, geometry is selected such as to customize the mesh or strut for specific clinical applications.

Step 2. Optionally, a polymer in dry form, e.g., in the form of a powder, is integrated into the plane of the magnesium mesh/strut (e.g., including within the pores or open spaces of the mesh and around the perimeter of the strut) through an optimized compression molding technique rendering an occlusive mesh/polymer composite. The polymer may be a single polymer or a co-polymer or a blend of various polymers. The polymer may be selected from poly(lactic-co-glycolic acid), e.g., low molecular weight poly(lactic-co-glycolic acid) and/or high molecular weight poly(lactic-co-glycolic acid), poly(lactic-co-glycolic acid), poly-L-lactic acid, poly-D-lactic acid, polyethylene glycol, and blends and mixtures thereof

Step 3. Polymer sheets are designed and manufactured using compression molding or like techniques. The sheets preferentially release magnesium to a periosteal side. The polymer may or may not be the same polymer as used in the foregoing Step 2.

Step 4. The polymer sheets (prepared in Step 3) are melted onto the magnesium mesh or strut produced in Step 1 above, or produced from the combination of Steps 1 and 2 above, using compression molding or like techniques. For example, a first polymer sheet is melted onto the upper surface of the mesh or strut, and/or a second polymer sheet is melted onto the lower surface of the mesh or strut. Each of the first and second polymer sheets may be composed of the same polymer. In certain embodiments, the first polymer sheet is composed of a first polymer and the second polymer sheet is composed of a different, second polymer. The resulting composite provides a magnesium/polymer barrier membrane that contains a magnesium mesh or strut support (e.g., a magnesium-reinforced polymer composite).

Alternately, a known lamination technique or like techniques may be employed to apply polymer sheet(s) or roll(s) to the magnesium mesh or strut produced in Step 1 above, or produced from the combination of Steps 1 and 2 above.

Step 5. In accordance with embodiments of the invention, a magnesium-reinforced polymer barrier membrane is produced, having optimized properties for handling in guided bone regeneration, and a design that is optimized for both bone regeneration and gingival tissue attachment.

According to the invention, bulk magnesium, e.g., in the form of a foil or sheet, is processed, e.g., by an expanded metal process or a laser cutting process, to produce a magnesium mesh or support strut. Polymer sheet is applied to the mesh or support strut, e.g., one sheet on each of an upper surface and/or a lower surface, e.g., to produce a magnesium/polymer barrier membrane.

Alternately, prior to applying the polymer sheet to the mesh or support strut, the magnesium mesh or support strut can be subjected to a molding process for integrating polymer powder with the mesh or support strut. Wherein the magnesium reinforcement is a mesh, the polymer powder can be integrated into the pores or open spaces formed within the mesh. For the support strut, the polymer powder can be formed/deposited around the perimeter of the strut. As a result, an occlusive magnesium mesh/polymer composite or an occlusive polymer sheet with magnesium strut is formed. Polymer sheet (Step 3) is then applied (Step 4) to each of the upper and/or lower surfaces of the occlusive magnesium mesh/polymer composite or occlusive polymer sheet with magnesium strut.

As mentioned herein, degrading metallic magnesium is more osteoconductive (enhances bone regeneration) than titanium. Thus, magnesium reinformcement and barrier membranes elicit faster bone regeneration in patients compared to currently used titanium products, and the design of the reinforcements and the barrier membranes can be optimized to leverage this phenomenon.

There are a variety of advantages demonstrated by the reinforcements and barrier membranes of the invention, including but not limited to, the release of magnesium from degrading metallic devices has the opportunity to greatly enhance bone regeneration when released in appropriate doses; and enhanced bone regeneration occurs preferentially at the periosteal interface. Thus, for example, the magnesium/polymer barrier membrane can be used in any periosteal contacting defect site in order to enhance bone regeneration as compared to existing implant devices.

Furthermore, the magnesium reinforcements and the magnesium-reinforced polymer barrier membranes can be fixed to a bone with various attachment mechanisms and techniques that are known in the art, such as bioabsorbable sutures, bioabsorbable tacks, minitacks or microtacks, or bioabsorbable screws, depending on the implantation site and size of the implant. Accordingly, corresponding holes can be made into the magnesium reinforcements and/or the magnesium-reinforced polymer barrier membranes to accommodate the various attachment mechanisms and techniques used.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention.

We claim:

1. A method of preparing a biodegradable, magnesium-reinforced polymer composite implant device, comprising:
selecting bulk magnesium;
selecting a polymer sheet;
processing the bulk magnesium to form a pre-determined geometry having a surface, employing a process selected from the group consisting of expanded metal processing and laser cutting; and
applying the polymer sheet onto the surface of the pre-determined geometry.

2. The method of claim 1, wherein the predetermined geometry is selected from the group consisting of a mesh, strut and strut-style support.

3. The method of claim 1, wherein the bulk magnesium is in a form selected from the group consisting of foil and sheet.

4. The method of claim 1, wherein the surface comprises an upper surface and a lower surface, and the applying the polymer sheet is onto one or more of the upper and lower surfaces.

5. The method of claim 4, wherein the applying the polymer sheet comprises melting a first polymer sheet onto the upper surface and a second polymer sheet onto the lower surface, wherein the first polymer sheet is the same as, or different from, the second polymer sheet.

6. The method of claim 5, further comprising obtaining a polymer in dry form and integrating the polymer in dry form into the pre-determined geometry, wherein the integrating is conducted prior to the melting of the first and second polymer sheets.

7. The method of claim 6, wherein the integrating comprises embedding the polymer in dry form within a plane of a magnesium mesh having pores or open spaces formed therein.

8. The method of claim 6, wherein the integrating comprises depositing the polymer in dry form along a perimeter of a magnesium strut.

9. The method of claim 1, wherein the applying the polymer sheet employs a process selected from the group consisting of compression molding and laminating.

10. A biodegradable, magnesium-reinforced polymer composite, comprising:
a magnesium framework having a surface, in a form selected from the group consisting of mesh, strut and strut-style support; and
a polymer sheet applied to the surface of the magnesium framework.

11. The composite of claim 10, wherein the mesh comprises polymer in dry form embedded in pores and openings formed in the mesh.

12. The composite of claim 10, wherein the strut comprises polymer in dry form formed along a perimeter of the strut.

13. The composite of claim 10, wherein the polymer sheet is selected from the group consisting of low molecular weight poly(lactic-co-glycolic acid), high molecular weight poly(lactic-co-glycolic acid), poly-L-lactic acid, poly-D-lactic acid, polyethylene glycol, and blends and mixtures thereof.

14. The composite of claim 10, wherein said composite is a biomedical implant device for applications selected from the group consisting of dental, craniofacial and orthopedic applications.

15. The composite of claim 14, wherein said applications include containing bone graft material and fixating complex craniofacial bone fractures.
